(19) United States
(12) Patent Application Publication
(10) Pub. No.: US 2006/0039968 A1
(43) Pub. Date: Feb. 23, 2006

(54) Gabapentin tablets and method for their preparation

(76) Inventors: Ramalingam Manikandan, Tamil Nadu (IN); Ashish Gogia, Delhi (IN); Sunilendu Bhushan Roy, Maharashtra (IN); Rajiv Malik, Delhi (IN)

Correspondence Address: RANBAXY INC., 600 COLLEGE ROAD EAST, SUITE 2100, PRINCETON, NJ, 08540, US

(21) Appl. No.: 10/530,592

(22) PCT Filed: Oct. 8, 2003
(86) PCT No.: PCT/IB03/04436

§ 371 Date: Apr. 7, 2005

(30) Foreign Application Priority Data
Oct. 8, 2002 (IN) 1023/DEL/2002

(51) Int. Cl. A61K 9/20

(52) U.S. Cl. 424/464; 264/109

(57) ABSTRACT

The present invention is generally directed to methods for preparing stable gabapentin tablets by wet granulation. A wet granulation method for preparing gabapentin tablets includes forming a mixture by dry mixing of a first portion of a binder with the gabapentin, one or more excipients, or a combination of the gabapentin and the one or more excipients; and adding a second portion of the binder to the mixture, wherein the second portion of the binder is in the form of a solution or dispersion.

### FIELD OF THE INVENTION

The invention is generally directed to stable gabapentin tablets prepared by wet granulation.

### BACKGROUND OF THE INVENTION

Gabapentin is an anti-epileptic drug indicated as adjunctive therapy in the treatment of partial seizures with and without secondary generalization in adults with epilepsy. Gabapentin exists in a crystalline form and exhibits poor compressibility and compactibility. These detrimental characteristics of gabapentin cause capping and lamination defects during compression of gabapentin into tablets.

Conventionally, these problems are overcome by incorporating compression aids in the formulation. However, the more excipients, such as compression aids, that are used in a composition the more expensive and time-consuming commercial production becomes. Moreover, increasing the amount of excipient increases the size of the tablet, which can result in overly large tablets, an undesirable result for pediatric use or for those patients who have difficulty in swallowing.

Moreover, including a large amount and/or number of excipients in a gabapentin formulation results in stability problems, such as degradation. For example, gabapentin has been found to degrade into lactam, resulting in a decrease in the potency of gabapentin over time. Because of the decrease in potency, it is necessary to avoid degradation of gabapentin over the shelf life of the product. Generally, shelf life of the product is two years from completion of manufacture. The level of degradation over the shelf life of the tablets can be determined by storing the product in closed containers for a three-month period at 40° C. and 75% relative humidity. It is generally accepted that tablets containing gabapentin should have no more than about 0.4% by weight of lactam, as determined by High Performance Liquid Chromatography (HPLC), at the end of this three-month period.

To combat the lactam formation and provide product stability, U.S. Pat. No. 6,054,482 discloses the importance of (a) starting with gabapentin raw material that contains 0.5% or less of corresponding lactam; (b) not allowing the anion of a mineral acid in the composition to exceed 20 ppm, and (c) using a specifically selected adjuvant that is not adverse to gabapentin stability.

In an attempt to achieve this, the patent discloses a method that includes hydrolyzing gabapentin with a semi-concentrated mineral acid and then converting gabapentin into solid pharmaceutical compositions containing hydroxypropylmethylcellulose (HPMC), polyvinylpyrrolidone, crospovidone, maize starch, cyclodextrin, talcum, co-polymer of dimethylaminomethacrylic acid and/or neutral methacrylic acid ester.

Another difficulty encountered in producing gabapentin tablets is that gabapentin is not amenable to traditional wet granulation techniques. Because the viscosity of the binder solution increases with the possible necessary increase in binder content, to apply a functional amount of binder for gabapentin the amount of solvent has to be increased. Increasing the amount of solvent, however, results in a wet granulation that is in a semi-liquid state and is not suitable for conventional drying methods. Therefore, to avoid the semi-liquid state, the wet granulation technique must be done in multiple stages in which a portion of binder solution is added, followed by drying, then the next portion of binder solution is added, and so forth. This becomes a time consuming and expensive process.

Purepac, the assignee of U.S. Pat. No. 6,294,198, has addressed this problem in the patent by using a spray-coating method in which a binder is dissolved in a solvent to form a binder solution that is then spray-coated on the drug particles. By using this method, substantially all of the solvent is evaporated as it is applied, leaving a film of binder around the drug particles. This process is conducted at or below room temperature.

### SUMMARY OF THE INVENTION

In one general aspect there is provided a wet granulation method for preparing stable gabapentin tablets. The wet granulation method includes: forming a mixture by dry mixing of a first portion of a binder with the gabapentin, one or more excipients, or a combination of the gabapentin and the one or more excipients; and adding a second portion of the binder to the mixture, wherein the second portion of the binder is in the form of a solution or dispersion.

Embodiments of the wet granulation method may include one or more of the following features. For example, the method may further include one or more of mixing the second portion of the binder with the mixture to form granules, drying the granules, mixing one or more excipients with the granules, and compressing into tablets.

The tablets may have a lactam content less than 0.1% by weight of gabapentin after one month of storage at 40° C. and 75% humidity, less than 0.2% by weight of gabapentin after two months of storage at 40° C. and 75% humidity, or less than 0.4% by weight of gabapentin after three months of storage at 40° C. and 75% humidity. In particular, the tablets may have a lactam content less than about 0.2% by weight of gabapentin after three months of storage at 40° C. and 75% humidity

The binder solution or dispersion may be prepared in water alone or in a mixture of water with one or more of ethanol, isopropyl alcohol, and acetone. The binder solution or dispersion may be prepared in water. The binder solution or dispersion may be prepared in a mixture of water and ethanol. The ratio of drug to binder may be between about 1:0.01 and about 1:1. The binder may be one or more of hydroxypropyl cellulose, hydroxypropyl methylcellulose, polyvinylpyrrolidone, copolyvidone, and sugars. In particular, the binder may be hydroxypropyl cellulose and/or copolyvidone.

The gabapentin may be the free base hydrated form, a monohydrate, or other pharmaceutically acceptable salt thereof. The gabapentin may have an anion of the mineral acid at about 100 ppm or less as calculated by chloride content. In particular, the anion of the mineral acid may be between about 20 and about 100 ppm.

The excipients mixed with the gabapentin or the granules may be one or more of disintegrants, fillers, stabilizers, lubricants, colorants, flavors, and glidants.

The disintegrant may be one or more of microcrystalline cellulose, sodium starch glycolate, crosslinked carboxy methylcellulose, and crospovidone. The disintegrant may be between about 0.5% w/w to about 15% w/w of the tablet.

The filler may be one or more of lactose, microcrystalline cellulose, mannitol, and dicalcium phosphate.

The stabilizer may be one or more of poloxamer, cremophor, anionic surfactants, cationic surfactants, and nonionic surfactants. The stabilizer may be about 0.1% w/w to about 10% w/w of the tablet.

The lubricant may be one or more of magnesium stearate, stearic acid, and stearyl fumarate.

The wet granulation method may further include coating the tablet. The coating may be one or more of a hydrophilic polymer, hydroxypropyl cellulose, hydroxypropyl methylcellulose, polyvinyl pyrrolidone, and polyvinyl alcohol. The coated tablet may have a friability of less than 1% w/w and an initial friability of less than about 0.1% w/w. The coated tablet may have a hardness of about 10 Kp to 30 Kp, and an initial hardness of between about 20 Kp and about 25 Kp.

In another general aspect there is provided a gabapentin tablet formed by wet granulation. The gabapentin tablet has a lactam content of less than 0.4% by weight of gabapentin after three months of storage at 40° C. and 75% humidity.

Embodiments of the tablet may include one or more of the features described above or following. For example, the wet granulation may include forming a mixture by dry mixing of a first portion of a binder with the gabapentin, one or more excipients, or a combination of the gabapentin and the one or more excipients; and adding a second portion of the binder to the mixture, wherein the second portion of the binder is in the form of a solution or dispersion.

In another general aspect there is provided a method of one or more of treating epilepsy, treating neuropathic pain, anticonvulsant therapy, treating post poliomyelitis pain, treating amyotrophic lateral sclerosis, controlling rapid cycling and mixed bipolar states, treating the pain of diabetic neuropathy, and as a prophylactic agent for patients with migraine headaches, the method including providing a gabapentin tablet prepared by wet granulation.

Embodiments of the tablet may include one or more of the features described above or following. For example, the wet granulation may include forming a mixture by dry mixing of a first portion of a binder with the gabapentin, one or more excipients, or a combination of the gabapentin and the one or more excipients; and adding a second portion of the binder to the mixture, wherein the second portion of the binder is in the form of a solution or dispersion. The gabapentin tablets may have a lactam content of less than 0.4% by weight of gabapentin after three months of storage at 40° C. and 75% humidity.

The details of one or more embodiments of the inventions are set forth in the description below. Other features, objects and advantages of the inventions will be apparent from the description and claims.

### DETAILED DESCRIPTION OF THE INVENTION

We have now discovered that stable gabapentin tablets can be prepared by a wet granulation method and, unlike the disclosure in U.S. Pat. No. 6,054,482, without having to limit use of gabapentin to a gabapentin having an anion of a mineral acid (calculated as chloride content) less than 20 ppm. The resulting tablets are not only free from capping and lamination defects but also have better hardness and are stable.

In one embodiment, the present invention relates to a wet granulation method for preparing stable gabapentin tablets, in which the tablets after three months of storage at 40° C. and 75% humidity have a lactam content less than 0.4% by weight of gabapentin.

This stability is provided by using a wet granulation method which includes dry mixing of a part of the binder with the drug, other excipients, or both; and then adding the rest of the binder in the form of a solution/dispersion. The addition of the binder in two portions is advantageous. First, the quantity of solvent used for preparing the binder solution is reduced to a minimum, which makes it possible to add binder solution in a single step. The two-portion addition also reduces the duration of exposure of gabapentin to the solvent, which can further reduce the likelihood of polymorph conversion and/or changes in crystal structure in gabapentin. Second, since the use of solvent is kept to a minimum, there is an improvement in the safety and environmental impact of the process.

Furthermore, the wet granulation method described herein also may be applied to other active drugs, and, in particular, those that have poor compressibility and compactibility.

In applying the method to gabapentin, any binder that is compatible with gabapentin may be used. For example, the binder may be selected from hydroxypropyl cellulose, hydroxypropylmethyl cellulose, polyvinyl pyrrolidone, copolyvidone, sugars, or a combination thereof. The binder may be dissolved or dispersed in a solvent such as water alone or a mixture of water and ethanol, isopropyl, alcohol and/or acetone. The concentration of binder in the solution will depend upon the components used and the desired viscosity. Typically, the drug to binder ratio will vary from about 1:0.01 to about 1:1. The binder solution/dispersion may be prepared by any method that permits dissolution of binder to produce a homogenous solution, mixture or dispersion, such that formulations may be prepared that will contain a uniform amount of the binder.

The gabapentin may be present as a free base, hydrated form, such as monohydrate, or any other pharmaceutically acceptable salt thereof. The amount of an anion of the mineral acid (calculated as chloride content) may vary up to about 100 ppm.

The other excipients in the formulation may be selected from one or more of disintegrants, fillers, stabilizers, lubricants, colorants, flavors and glidants.

The disintegrant may be one or more of microcrystalline cellulose, sodium starch glycolate, crosslinked carboxy methylcellulose, crospovidone, other suitable disintegrants, or a combination thereof. The disintegrant may be present intragranularly, as well as extragranularly. The disintegrant may be used at a concentration of about 0.5% w/w to about 15% w/w of the tablet.

The fillers may be one or more of any conventional filler, such as lactose, microcrystalline cellulose, mannitol, dicalcium phosphate, other suitable fillers, or a combination thereof.

The stabilizer may be one or more of poloxamer, cremophor, other anionic, cationic, nonionic surfactants, or a combination thereof. The stabilizer may be used in concentration of between about 0.1% w/w to about 10% w/w of tablet.

The lubricant may be one or more of magnesium stearate, stearic acid, sodium stearyl fumarate, other suitable lubricants, or combinations thereof.

The method may be carried out using the following steps:
(i) Gabapentin is mixed with one or more disintegrants in a mixer.
(ii) The binder is divided into two portions, one portion is mixed with the gabapentin-disintegrant mixture and the remaining portion is dissolved in a sufficient quantity of granulating solvent to prepare a binder solution.
(iii) The binder solution is then mixed with the gabapentin-disintegrant-binder mixture of step (ii) in a low shear mixer.
(iv) The granules of step (iii) are dried in a fluidized bed dryer.
(v) The dried granules are mixed with rest of the excipients, e.g., stabilizers, fillers, glidants, disintegrants (extragranular) and lubricants and compressed into tablets using appropriate tooling.

For ease of swallowing and to enhance the aesthetic appeal, it may be desirable to coat the tablet as an optional step. The coating may be made of one or more hydrophilic polymers such as hydroxypropyl cellulose, hydroxypropyl methylcellulose, polyvinyl pyrrolidone and polyvinyl alcohol.

The tablets prepared by the above method have a hardness of about 10 Kp to about 30 Kp and a friability of less than 1% w/w.

The lactam content of the gabapentin tablet made by the above method does not exceed 0.4% by weight of gabapentin after storage for three months at 40° C. and 75% relative humidity.

The following examples are provided for the purpose of illustrating the present invention and are not intended to limit the scope of the invention.

### EXAMPLES

Core

Quantity (mg)

| Ingredient | Example 1 | Example 2 |
|---|---|---|
| Intragranular | | |
| Gabapentin | 800 | 800 |
| Hydroxypropyl cellulose-L (HPC-L) | 40 | 40 |
| Crospovidone | 22 | — |
| Extragranular | | |
| Crospovidone | 22 | 44 |
| Corn starch | 60 | — |
| Poloxamer | 11 | 11 |
| Dicalcium phosphate | 68 | — |
| Mannitol | 110 | 178 |
| Talc | 11 | 11 |
| Magnesium stearate | 16 | 16 |

Method:

### Example 1

Gabapentin, HPC-L (half quantity) and crospovidone are mixed in a rapid mixed granulator and granulated with a HPC-L solution/dispersion in purified water and dried in a fluid bed dryer. The resulting dried granules are mixed with the extragranular excipients, i.e., crospovidone, corn starch, poloxamer, dicalcium phosphate and mannitol, in a low shear blender for 15 minutes. The resulting blend is mixed with talc and magnesium stearate in a low shear blender for 10 minutes and compressed into tablets using appropriate tooling.

### Example 2

Gabapentin and HPC-L (half quantity) are mixed in a rapid mixer granulator and granulated with a binder solution (i.e., the solution of the rest of the quantity of HPC-L in purified water) and dried in a fluid bed dryer. The resulting dried granules are mixed with the extragranular excipients, i.e., crospovidone, poloxamer and mannitol, in a low shear blender for 15 minutes. The resulting blend is finally mixed with talc and magnesium stearate in a low shear blender for 10 minutes and compressed into tablets using appropriate tooling.

The tablets made as per the above examples are coated with a coating having the following composition:

Coating formula:
Hydroxypropylcellulose: 15 mg
Talc: 15 mg
Purified water: q.s.

The tablets of Example 2 were subjected to accelerated studies for three months at 40° C. and 75% relative humidity (RH). The resulting stability, friability and hardness data are shown in Tables 1 and 2.

TABLE 1
Stability data of gabapentin tablets
subjected to accelerated studies.

| | | 1 M/40° C./ | 2 M/40° C./ | 3 M/40° C./ |
|---|---|---|---|---|
| | Initial | 75 % RH | 75% RH | 75% RH |
| Gabapentin | 99.51 | 97.02 | 101.4 | 99.04 |
| (% w/w) | | | | |
| Gabapentin | N.D* | 0.027 | 0.139 | 0.198 |
| lactam | | | | |
| derivative | | | | |
| (% w/w) | | | | |

*N.D.—Not detected

TABLE 2
Friability and Hardness data

| | Friability | Hardness |
|---|---|---|
| Tablet | (% w/w) | Range (Kp) |
| Uncoated tablets | 0.25 | 16-18 |
| Coated Tablets (initial) | 0.03 | 20-24 |
| Coated Tablets (One month | 0.00 | 22-27 |
| at 40° C./75% RH) | | |
| Coated Tablets (Two months | 0.10 | 19-24 |
| at 40° C./75% RH) | | |
| Coated Tablets (Three months | 0.04 | 20-22 |
| at 40° C./75% RH) | | |

As can be seen from Tables 1 and 2, the tablets have very low levels of the lactam and acceptable friability and hardness values initially and after storage at 40° C. and 75% relative humidity for up to three months.

While several particular forms of the inventions have been described, it will be apparent that various modifications and combinations of the inventions detailed in the text can be made without departing from the spirit and scope of the inventions. For example, the gabapentin tablets described herein can be used for any approved or unapproved use for which gabapentin provides therapeutic benefit. These uses include but are not limited to: (1) as an adjunctive therapy in the treatment of partial seizures with and without secondary generalization in adults with epilepsy; (2) as an anticonvulsant used to control various types of seizures in the treatment of epilepsy; (3) for post poliomyelitis pain and amyotrophic lateral sclerosis; (4) for controlling rapid cycling and mixed bipolar states in people who have not received adequate relief from carbamazepine and/or valproate; (5) treatment for the pain of diabetic neuropathy; (6) reducing the pain from chronic neuropathic pain (e.g., due to damaged nerves) while also reducing sleep disturbances and improving mood and enhancing patients' quality of life; and (7) as a prophylactic agent for patients with migraine headaches. Reports indicate that gabapentin also can be used for the treatment of pain (neuropathies, neuralgias, fibromyalgia, chronic, back, headache, migraine), bipolar affective disorder, epilepsy, restless leg, multiple sclerosis, anxiety, and behavior disorders. The gabapentin drug products made according to the methods disclosed herein also can be used for these indications and treatments. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

We claim:

1. A wet granulation method for preparing a stable gabapentin tablet, the wet granulation method comprising:
forming a mixture by dry mixing of a first portion of a binder with the gabapentin, one or more excipients, or a combination of the gabapentin and the one or more excipients; and
adding a second portion of the binder to the mixture, wherein the second portion of the binder is in the form of a solution or dispersion.

2. The wet granulation method of claim 1, further comprising mixing the second portion of the binder with the mixture to form granules.

3. The wet granulation method of claim 2, further comprising drying the granules.

4. The wet granulation method of claim 3, further comprising mixing one or more excipients with the granules.

5. The wet granulation method of claim 3, further comprising compressing into tablets.

6. The wet granulation method of claim 5, wherein the tablets have a lactam content less than 0.1% by weight of gabapentin after one month of storage at 40° C. and 75% humidity.

7. The wet granulation method of claim 5, wherein the tablets have a lactam content less than 0.2% by weight of gabapentin after two months of storage at 40° C. and 75% humidity.

8. The wet granulation method of claim 5, wherein the tablets have a lactam content less than 0.4% by weight of gabapentin after three months of storage at 40° C. and 75% humidity.

9. The wet granulation method of claim 8, wherein the tablets have a lactam content less than about 0.2% by weight of gabapentin after three months of storage at 40° C. and 75% humidity

10. The wet granulation method of claim 1, wherein the binder solution or dispersion is prepared in water alone or in a mixture of water with one or more of ethanol, isopropyl alcohol, and acetone.

11. The wet granulation method of claim 10, wherein the binder solution or dispersion is prepared in water.

12. The wet granulation method of claim 10 wherein the binder solution or dispersion is prepared in a mixture of water and ethanol.

13. The wet granulation method of claim 1, wherein the ratio of drug to binder is beween about 1:0.01 and about 1:1.

14. The wet granulation method of claim 1, wherein the wherein the binder comprises one or more of hydroxypropyl cellulose, hydroxypropyl methylcellulose, polyvinylpyrrolidone, copolyvidone, and sugars.

15. The wet granulation method of claim 14, wherein the binder comprises hydroxypropyl cellulose.

16. The wet granulation method of claim 14, wherein the binder comprises copolyvidone.

17. The wet granulation method of claim 1, wherein gabapentin comprises the free base hydrated form, a monohydrate, or other pharmaceutically acceptable salt thereof.

18. The wet granulation method of claim 1, wherein the gabapentin has an anion of the mineral acid at about 100 ppm or less as calculated by chloride content.

19. The wet granulation method of claim 18, wherein the anion of the mineral acid is between about 20 and about 100 ppm.

20. The wet granulation method of claim 1, wherein the excipients comprise one or more of disintegrants, fillers, stabilizers, lubricants, colorants, flavors, and glidants.

21. The wet granulation method of claim 4, wherein the excipients comprise one or more of disintegrants, fillers, stabilizers, lubricants, colorants, flavors, and glidants.

22. The wet granulation method of claim 20, wherein the disintegrant comprises one or more of microcrystalline cellulose, sodium starch glycolate, crosslinked carboxy methylcellulose, and crospovidone.

23. The wet granulation method of claim 20, wherein the disintegrant comprises between about 0.5% w/w and about 15% w/w of the tablet.

24. The wet granulation method of claim 20, wherein the disintegrant comprises crospovidone.

25. The wet granulation method of claim 20, wherein the filler comprises one or more of lactose, microcrystalline cellulose, mannitol, and dicalcium phosphate.

26. The wet granulation method of claim 20, wherein the stabilizer comprises one or more of poloxamer, cremophor, anionic surfactants, cationic surfactants, and nonionic surfactants.

27. The wet granulation method of claim 20, wherein the stabilizer comprises between about 0.1% w/w to about 10% w/w of the tablet.

28. The wet granulation method of claim 20, wherein the lubricant comprises one or more of magnesium stearate, stearic acid, and stearyl fumarate.

29. The wet granulation method of claim 5, further comprising coating the tablet.

30. The wet granulation method of claim 29, wherein the coating comprises one or more of a hydrophilic polymer, hydroxypropyl cellulose, hydroxypropyl methylcellulose, polyvinyl pyrrolidone, and polyvinyl alcohol.

31. The wet granulation method of claim 29, wherein the coated tablet has a friability of less than 1% w/w.

32. The wet granulation method of claim 29, wherein the coated tablet has an initial friability of less than about 0.1% w/w.

33. The wet granulation method of claim 29, wherein the uncoated tablet has a hardness of between about 10 Kp to about 30 Kp.

34. The wet granulation method of claim 29, wherein the uncoated tablet has an initial hardness of between about 20 Kp and about 25 Kp.

35. A gabapentin tablet formned by wet granulation, the gabapentin tablets having a lactam content of less than 0.4% by weight of gabapentin after three months of storage at 40° C. and 75% humidity.

36. The gabapentin tablet of claim 35, wherein the wet granulation comprises forming a mixture by dry mixing of a first portion of a binder with the gabapentin, one or more excipients, or a combination of the gabapentin and the one or more excipients; and adding a second portion of the binder to the mixture, wherein the second portion of the binder is in the form of a solution or dispersion.

37. A method of one or more of treating epilepsy, treating neuropathic pain; providing an anticonvulsant therapy, treating post poliomyelitis pain, treating amyotrophid lateral sclerosis, controlling rapid cycling and mixed bipolar states, treating the pain of diabetic neuropathy, and as a prophylactic agent for patients with migraine headaches, the method comprising providing a gabapentin tablet prepared by wet granulation.

38. The method of claim 37, wherein the wet granulation comprises forming a mixture by dry mixing of a first portion of a binder with the gabapentin, one or more excipients, or a combination of the gabapentin and the one or more excipients; and adding a second portion of the binder to the mixture, wherein the second portion of the binder is in the form of a solution or dispersion.

39. The method of claim 37, wherein the gabapentin tablets have a lactam content of less than 0.4% by weight of gabapentin after three months of storage at 40° C. and 75% humidity.
